(19) United States
(12) Patent Application Publication
(10) Pub. No.: US 2003/0092178 A1
(43) Pub. Date: May 15, 2003

(54) Cell culture incubator with dynamic oxygen control

(75) Inventor: Randy Yerden, Redfield, NY (US)

Correspondence Address: Bernhard P. Molldrem, Jr., 333 East Onondaga Street, Syracuse, NY, 13202, US

(73) Assignee: BioSpherix, Ltd.

(21) Appl. No.: 09/999,430

(22) Filed: Nov. 15, 2001

(51) Int. Cl. C12N 5/02

(52) U.S. Cl. 435/375; 435/383

(57) ABSTRACT

During growth of tissue culture in a controlled atmsophere incubator the oxygen (another environmental gas) is dynamic controlled according to an oxygen profile (or other profile). Gaseous oxygen concentration is deliberately changed over time in a precise and reproducible way, with other parameters being held constant, or with other controlled parameters being changed simultaneously or sequentially. Oxygen profile is developed based on the amount of gas phase oxygen needed to maintain the dissolved gas in the culture medium at a desired level. A gas controller is programmed with the gas profile, and the oxygen is dynamically controlled according to the oxygen profile. The specific sequence and timing of changes in gaseous oxygen concentration that best indirectly regulate the desired dissolved oxygen concentration in the culture media inside open culture vessels that are placed inside the incubator.

### BACKGROUND OF THE INVENTION

This invention concerns tissue or cell culture chambers such as incubators and similar chambers in which open culture vessels, i.e., culture dishes, are provided with a controlled environment, and is more particularly concerned with techniques for controlling the atmospheric parameters inside the incubation chamber, such as the concentration of a gas. The invention is more specifically directed to techniques for creating dynamic instead of static atmospheric conditions inside cell culture incubators for the purposes of achieving better growth, or achieving better viability over longer periods of time with or without growth, or achieving better simulation in vitro (in the dish) of those natural conditions that occur in vivo (in the body), including both normal and pathologic conditions.

Cells and tissue taken from a multi-cellular organism, for example, can be cultured outside the body in various liquid and semi-liquid media contained in culture vessels (in vitro) by simulating as closely as possible the environment to which these cells would normally be exposed while inside the body (in vivo). Many physical, chemical, and biological variables make up this critical environment. Generally, as long as all the essential variables remain within some physiologic range, cells and tissue in vitro will grow and differentiate and remain viable.

Since different cell types and different culture preparations of the same cell type have different requirements, the first step in the advancement of cell culture technology is to discover the entire set of essential variables for the given culture. The next step is to determine the optimum levels for each variable at every point in time over the course of that culture. Once these are accomplished, the final challenge is to simultaneously maintain each and every essential variable at or near its optimum level, at or near the proper time in the course of the culture, over the entire duration of the culture.

Oxygen concentration is one of the most universally important variables. Oxygen is the main electron sink driving respiration. Oxygen is also a critical substrate in many enzymatic oxygenation and oxidation reactions. For these reasons, oxygen must be maintained at sufficiently high levels in the media bathing the cells for the cells to function properly.

On the other hand, oxygen is also a concentration dependent toxin. It spontaneously breaks down and is metabolically broken down into free radicals and other highly reactive moieties, which are so unstable that they react indiscriminately with and damage whatever molecular structures happen to be nearby. For this reason, oxygen must be maintained at sufficiently low levels, so that the toxic byproducts of oxygen are not formed at a rate which will overwhelm the normal cellular antioxidant defense mechanisms.

Cell cultures are dynamic living entities that grow and evolve, and their metabolic activity changes over time. For one example, the oxygen consumption rate of a rapidly growing cell population in culture rises over time as the population size and density increases. Cell culture technologies that directly control oxygen in the culture vessel, such as bio-reactors, have the ability to respond to changes in oxygen demand. In response to an increase in oxygen demand, a direct control system will increase the oxygen supply rate to the culture in order to keep the oxygen concentration in the media from becoming dangerously low. Or it can change in the other direction: Overall oxygen consumption rate can decrease over time in culture, for instance, as a non-growing cell population differentiates from a high metabolic state to a more dormant state. Again, with direct control of oxygen in the culture vessel, the response would be to decrease the oxygen supply rate in order to keep the oxygen concentration in the media from becoming dangerously high. One example, among many others, of a direct oxygen control culture system is described in Pippen et al. U.S. Pat. No. 3,772,176.

However, many cells and tissues are cultured in culture vessels in which there is no direct control of oxygen. Instead the culture vessels are kept open and are placed inside controlled atmosphere chambers, typically called incubators, which create and contain a certain gas oxygen concentration considered to be at least sufficient if not optimal (in addition to a few other variables such as temperature, carbon dioxide concentration, and relative humidity). Oxygen is thereby indirectly controlled inside the culture vessel only as a result of the culture vessel being open with and contiguous to the common gas oxygen atmosphere inside the incubator. In some incubators oxygen is actively controlled at some specific level, and in other incubators it is not. With no control, oxygen is always fixed at just under the ambient air oxygen level. In any case, the typical objective of such incubators is to maintain the gas oxygen concentration constant throughout the incubator and constant over the time required for the cultivation of the culture. One example of a current state of the art controlled atmosphere cell culture incubator is described in Hugh et al. U.S. Pat. No. 5,792,427.

In any open culture vessel within such controlled atmosphere incubators, oxygen can only be supplied to the cells in the liquid media by diffusion of oxygen into the liquid phase from that part of the gas phase which is in contact with the liquid. If the oxygen concentration in the gas phase is constant, that means the oxygen supply rate is fixed. With a fixed oxygen supply rate and with no ability to respond to changes in oxygen demand in any of the culture vessels placed within them, common laboratory cell culture incubators with static oxygen atmospheres are severely limited in their ability to keep oxygen at optimum levels for very long over the course of any given culture. For example, once the oxygen demand rate exceeds the fixed supply rate in a culture with a rapidly increasing cell population density, the culture will not be able to get enough oxygen and can die of oxygen starvation.

Consequently, to keep rapidly growing cultures alive over long periods, one common procedure is to keep the cell population density within a certain range which is compatible with the fixed oxygen supply rate afforded by the incubator oxygen tension. This is carried out by routine subculture, before the cell population density reaches a dangerously high level that would lead to oxygen deprivation. Such a procedure involves considerable labor and materials, may increase chance of contamination, and limits the range of different culture preparations that can be grown.

However, since current incubator technology with static oxygen does not otherwise recognize that oxygen requirements change over time, there are only two other options when using current state of the art incubators. One option is to change the oxygen concentration in the incubator manually at appropriate times. The other is to maintain multiple incubators with different oxygen tensions and physically move the open culture vessels from incubator to incubator at the appropriate times. Both these options assume the incubators have oxygen control, and of course, many do not. Obviously, in incubators without oxygen control, neither option is available. Yet, even with those incubators that do have the ability to control oxygen, both options are inherently difficult to carry out. Manually changing the oxygen level in an incubator requires rigorous scheduling, which is both inconvenient and subject to human error. Moving cultures among multiple incubators has the same problems. In addition, physically handling the cultures increases chance of contamination, and also requires a considerable investment in equipment and space for all the incubators that would be needed.

Outside of incubators, the only other common way to control the atmosphere of open dish cultures is to place them inside a sealable container, and then flush the container with a prepared gas mixture to condition the gas phase. Once conditioned, the containers are sealed and placed in a temperature controlled chamber. It would be possible to dynamically change the oxygen concentration over the course of a culture by the use of numerous gas mixtures with different oxygen levels, and flush each appropriate mixture at the appropriate time. Yet this method is fraught with even more difficulty. It presents all the same problems of the aforementioned manual incubator procedures, but requires even more labor.

Moreover, none of these current options provide a definitive method for determining what the optimum gas oxygen levels are for different open dish culture preparations over the entire course of their culture period.

In view of the above noted problems and deficiencies of current controlled atmosphere incubators with static oxygen levels, there is a need for incubators and culture chambers for the culture of cells and tissue in open culture vessels in which the gaseous oxygen concentration is deliberately and automatically changed over time in a precise and reproducible way. Additionally, there is a need for apparatus and a technique for determining the optimal gas oxygen levels and timing that will be required for any given open dish culture preparation.

### OBJECTS AND SUMMARY OF THE INVENTION

Accordingly, it is an object of the present invention to provide an incubator or controlled atmosphere chamber for open dish cultures which, instead of being limited with static controlled conditions, can carry out precise and deliberate automated changes in gas phase oxygen concentration. It will be able to change oxygen concentration while holding all other controlled parameters constant, or while one or more other controlled parameters are changed simultaneously or sequentially as well.

Another object is to control the atmosphere in an incubator for a cell and tissue culture in which the gaseous oxygen concentration is deliberately changed over time in a precise and reproducible way, while all the other parameters are held constant, or while one or more other controlled parameters are changed simultaneously or sequentially.

A related object is to determine the specific sequence and timing of changes in the gaseous oxygen concentration that will best indirectly regulate the desired dissolved oxygen concentration in the culture media inside open culture vessels that are placed inside the incubator.

Moreover, it is an object to employ specific gas oxygen profiles in such chambers for the purpose of achieving better growth over longer periods of time, or achieving better viability with or without growth over longer periods of time, or achieving better simulation in vitro (in the dish) of conditions that occur naturally in vivo (in the body) in the open dish cultures that are incubated in such chambers. That is, it is not only on object to create conditions of growth of healthy tissue, but in many cases it is desired to simulate cell pathologies. Gas oxygen profiles are defined here as a linear sequence of time segments of different or similar duration, each with different or similar gas oxygen concentration setpoints which can be controlled sequentially once, or cycled a defined number of times, or cycled continuously. Dissolved oxygen profiles, in contrast, are a linear sequence of timed dissolved oxygen setpoints.

It is another object of the invention to provide methodology for determining a specific gas oxygen profile in the culture chamber that will best indirectly regulate the desired dissolved oxygen concentration in the culture media inside the open culture vessels which contain certain cell or tissue culture preparations that are to be cultured inside the chamber.

In all embodiments of the invention, a servo control loop regulates the oxygen concentration. This oxygen control loop may be designed to work alone in some embodiments. In others, it may be designed to work in conjunction with one or more additional control loops used to control other variables in the same chamber, such as temperature or carbon dioxide. The oxygen control loop may be designed to control oxygen directly in the culture chamber, or indirectly in a secondary gas mixing apparatus and then circulate oxygen controlled gas to the culture chamber.

In typical servo fashion, this oxygen control loop utilizes the input or feedback from an oxygen sensor to activate one or more appropriate outputs in order to reduce towards zero the difference between the oxygen sensor reading and the oxygen setpoint. Setpoints are defined here as the oxygen concentration desired by the operator. They can be gas oxygen setpoints or dissolved oxygen setpoints. Setpoints can be entered manually one at a time by the operator, or a series of setpoints can be provided to the control loop automatically at times as specified by an oxygen profile. The outputs consist of one or more actuators of any suitable type that can increase the gas oxygen concentration in the chamber, and others that can decrease the gas oxygen concentration in the chamber. However, the input or feedback to this control loop can be provided by either a gas oxygen sensor or a dissolved oxygen sensor. Gas oxygen sensors monitor the oxygen concentration in the gas phase inside the incubation chamber, and can be of any suitable type. Gas sensors provide feedback to control gas setpoints. Dissolved oxygen sensors monitor the oxygen concentration in the liquid media contained in one or more of the open culture vessels incubating inside the chamber, and can be of any suitable type compatible with such culture vessels. Dissolved oxygen sensors serve as feedback to control dissolved oxygen setpoints in the liquid.

The invention contemplates several different embodiments and several different methods of operation depending on the particular needs of the operator and various limitations. Those limitations may be imposed by economic constraints, imposed by specific requirements of certain culture preparations, or imposed by the particular technology used to monitor the dissolved oxygen concentration in individual culture vessels.

Some embodiments may require only a gas oxygen sensor and may not need a dissolved oxygen sensor at all. If the gas oxygen profile is known, or if it is possible to empirically determine a suitable gas oxygen profile, it may be more economical and practical to employ in some culture chambers only a gas oxygen sensor to provide the feedback to the oxygen control loop. For example, just by knowing that the oxygen demand increases over time in a rapidly growing culture, it may be possible to achieve a substantial improvement over an incubator with a static oxygen level by simply controlling a gas oxygen profile that starts out at a low oxygen level and gradually increases the oxygen level over the duration of the culture.

Other embodiments may require only a dissolved oxygen sensor and may not need a gas oxygen sensor. Whenever the particular technology utilized for sensing the dissolved oxygen concentration is not disruptive in any significant way to the culture in which it is measuring the oxygen, it should be possible to directly control the oxygen concentration in the media inside the open culture vessel at any single dissolved oxygen setpoint or series of setpoints in a profile, with no particular need for a gas oxygen sensor at all. For example, it may be desirable to keep the dissolved oxygen tension constant at some hypoxic, normoxic, or hyperoxic level in a rapidly growing culture. With a fixed dissolved oxygen setpoint, the gas oxygen level would be adjusted higher and higher over the course of such a culture in order to keep up with increased It oxygen demand and thereby maintain a constant oxygen tension in the media. Such direct control of the dissolved oxygen tension could compensate in real time for variations in demand is over the course of the culture, and variations in demand among different culture preparations. In this regard it should be possible to achieve a substantial improvement over a static oxygen level incubator by letting the oxygen control loop blindly profile oxygen in the gas phase.

In circumstances where the process of sensing the dissolved oxygen concentration in the liquid media may be disruptive or potentially disruptive, it will be necessary to use a surrogate culture that is prepared to be similar to the culture of interest, or an identical sample of the same culture preparation which is expendable. Direct control of the dissolved oxygen in such a witness dish creates the gas oxygen profile which is optimal for the separate undisturbed neighboring culture of interest.

In one preferred embodiment at least one dissolved oxygen sensor and one gas oxygen sensor are utilized simultaneously in the same culture chamber. A primary aspect of this embodiment of the invention is that either the gas oxygen sensor or the liquid oxygen sensor can provide the input or feedback to the oxygen servo control loop. Furthermore, it will be possible to switch back and forth between the two at the beginning of each new culture, or at any point in time over the course of a culture. Switching can be done manually or automatically. Regardless of which type of oxygen sensor is serving as the input to the control loop, the oxygen readings from both types of sensors are continuously monitored and recorded, and preferably but not necessarily displayed in real time. Setpoints are differentiated as either dissolved oxygen setpoints or gas oxygen setpoints. Of course, such a dual sensor embodiment could be operated in either of the other single sensor modes described just above.

The invention further contemplates that simultaneous dissolved oxygen and gas oxygen sensing with switchable feedback provides the unique ability to determine the specific gas oxygen profile in the chamber that will best indirectly regulate the desired dissolved oxygen concentration in any given culture preparation. The procedure would first entail utilizing the dissolved oxygen sensor as the input to the oxygen control loop and directly controlling the oxygen concentration in the liquid media. As dissolved oxygen setpoints are controlled, the gas oxygen levels that result are recorded as a function of time. These gas oxygen levels are then converted to gas oxygen setpoints in a gas oxygen profile, which can then be recreated anytime. Recreating the same dynamic atmosphere over identical culture preparations will indirectly regulate the same dissolved oxygen concentrations. This can be verified in the same chamber by switching to the gas oxygen sensor for feedback to the loop, and controlling that same recorded gas oxygen profile during the repeat incubation of an identical culture preparation while monitoring the dissolved oxygen.

In an alternative method, the dissolved oxygen measurements are simply recorded over the time that a specific gas oxygen profile is controlled in the incubator. This allows an empirical approach to the optimization of a profile by testing different gas profiles and ascertaining how close each different profile comes to achieving the desired levels of dissolved oxygen.

Determination of the proper gas profile may be routine procedure for each new culture preparation so that subsequently it may be successfully cultured in chambers without dissolved oxygen sensors. It may be required for certain culture preparations in which the process of sensing the dissolved oxygen concentration is disruptive or potentially disruptive.

Oxygen profiling is a primary importance, but other embodiments could include profiling of other bio-active gases, such as CO₂, CO, NO, Et, CH₄, NH₃, etc., and/or profiling of temperature, although temperature profiling is common in other applications. The tissue or cells may be human, other animal, plant, or microbial.

The above and many other objects, features, and advantages of this invention will be more fully appreciated from the ensuing description of preferred embodiments, which is to be read in conjunction with the accompanying Drawing.

### BRIEF DESCRIPTION OF DRAWING

FIG. 1 is a functional schematic of a culture chamber and all associated components for carrying out the processes common to all different physical embodiments of this invention.

FIG. 2 is a schematic illustrating one physical embodiment described as a retrofit embodiment.

FIG. 3 is a schematic illustrating another physical embodiment described as a sub-chamber embodiment.

FIG. 4 is a schematic illustrating another physical embodiment described as a remote conditioning embodiment.

FIG. 5 is a schematic illustrating another physical embodiment described as an integrated stand-alone single chamber incubator embodiment.

FIG. 6 is a schematic illustrating another physical embodiment described as an integrated stand-alone multi-chamber incubator embodiment.

### DETAILED DESCRIPTION OF THE PREFERRED EMBODIMENT

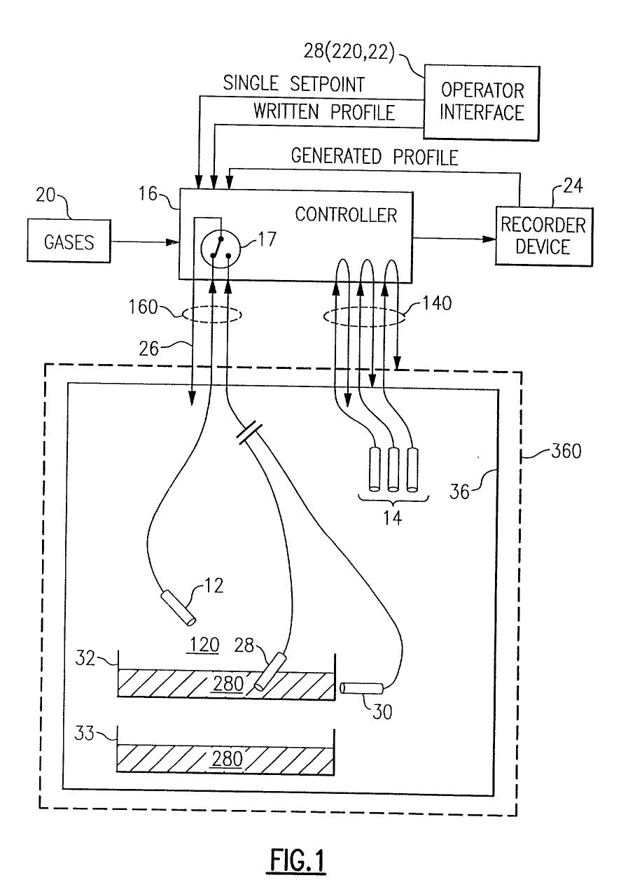

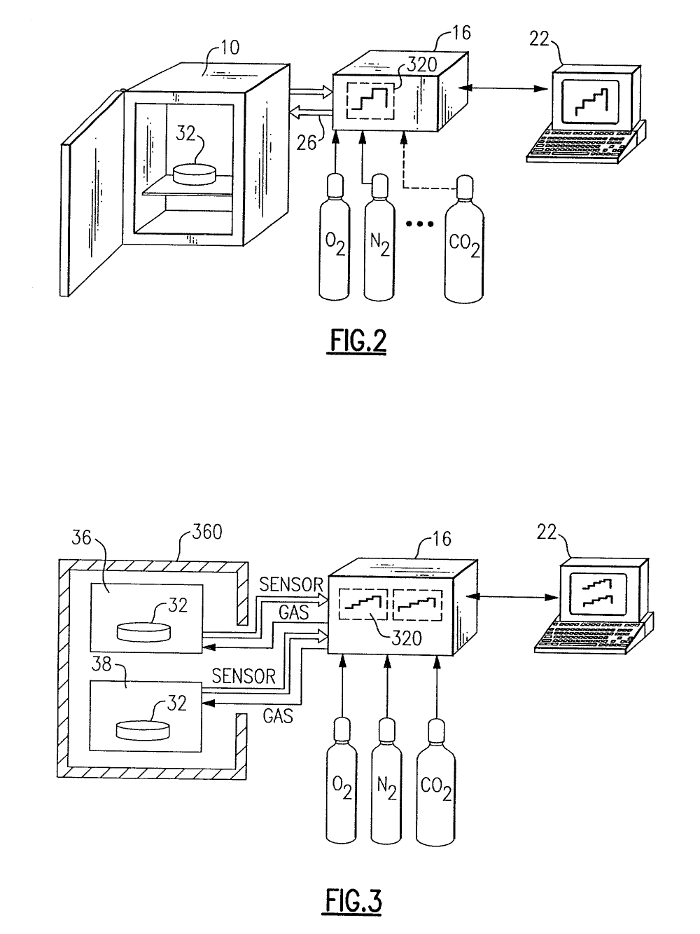

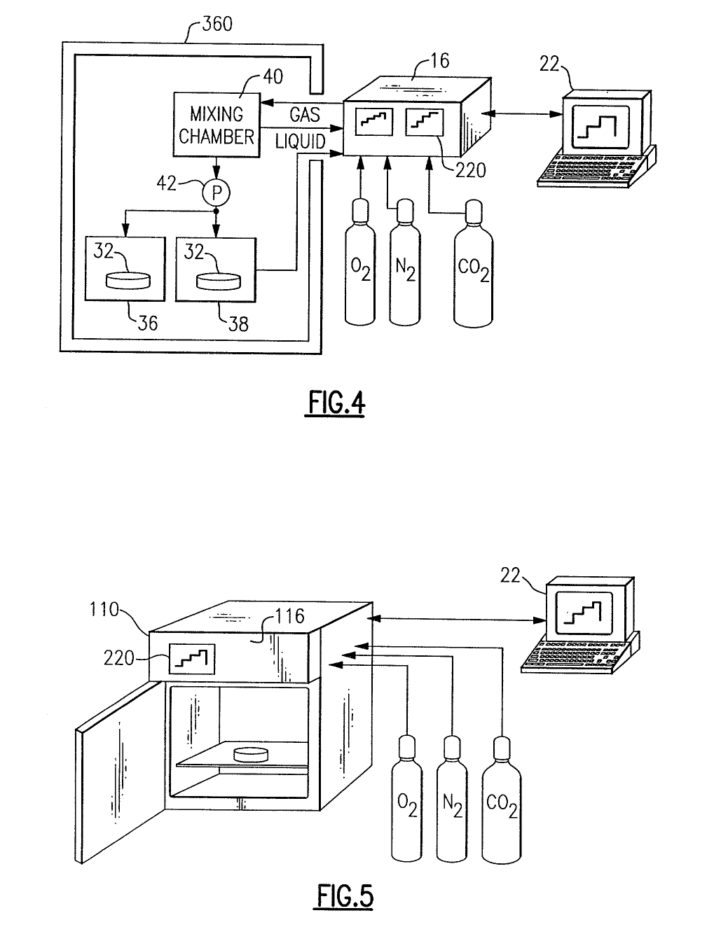

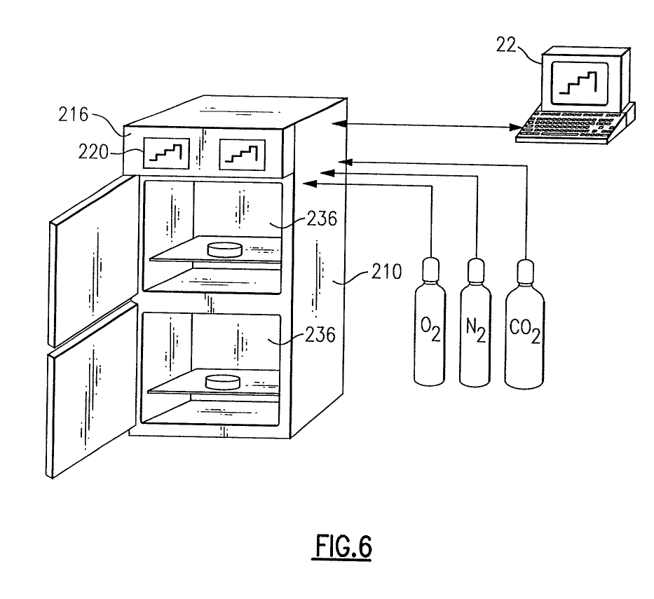

With reference now to the Drawing, FIG. 1 is a block diagram and schematic of the functional components of the invention common to various physical embodiments as depicted in FIGS. 2-6. An oxygen profile controller 16 controls gas oxygen tension inside each of one or more culture chambers 36 which contains the cell or tissue cultures in open culture vessels 32 and 33. The culture chamber 36 may be a stand-alone chamber such as any commercially available incubator 10 as depicted in FIG. 2, or an integrated single chamber incubator 110 as depicted in FIG. 5, or an integrated multiple chamber incubator 210 as depicted in FIG. 6. Alternatively, the culture chamber 36 may be a sub-chamber inside another host chamber 360 as depicted in FIGS. 3 and 4, and multiple culture chambers 36 and 38 can be sub-chambers inside the same or different host chambers 360.

In all embodiments the controller 16 controls oxygen via an oxygen servo control loop 160 in each culture chamber 36 according to feedback from either a gas oxygen sensor 12 monitoring gas oxygen tension 120, or a liquid oxygen sensor 28, 30 monitoring dissolved oxygen tension 280 in the culture media contained in at least one witness culture vessel 32 in that culture chamber 36. The dissolved oxygen 280 in a witness dish 32 will be representative of the dissolved oxygen in all other non-monitored culture vessels 33 of the same culture preparation since their oxygen is supplied from the same common gas oxygen tension 120. The dissolved oxygen sensor may be an invasive sensor 28 or a non-invasive sensor 30. An invasive oxygen sensor 28 is one that is immersed into the culture media, such as an electrochemical oxygen sensor. A non-invasive oxygen sensor 30 is one that measures from outside the culture vessel 32, such as an optical oxygen sensor. Depending on economic and technical constraints and needs of the operator, the oxygen servo control loop 160 in some culture chambers 36 will require only a gas oxygen sensor 12, some will require only a dissolved oxygen sensor 28 or 30, and some will require both. Those configured with both gas oxygen and dissolved oxygen sensors will have a switch 17 that will allow the operator to select which sensor is to provide the feedback to the oxygen control loop 160.

The controller 16 can actuate control of the oxygen level via the oxygen servo control loop 160 in each culture chamber 36 most simply by the infusion of gases 26 from continuous supplies of those gases 20 in order to displace by dilution the previous atmosphere. Oxygen or oxygen-enriched gas can be infused to raise the oxygen level. Oxygen free gas such as nitrogen or oxygen-depleted gas can be infused to lower the oxygen level. However, any other suitable method of changing the oxygen level, such as generating or scrubbing oxygen may be utilized as well. Regardless of whether a gas oxygen sensor 12 or a dissolved oxygen sensor 28 or 30 is providing the feedback to the oxygen control loop 160, the actuation of oxygen control is the same, that is, the raising or lowering of the chamber gas oxygen tension 120.

Oxygen control setpoints are designated as either gas oxygen setpoints or dissolved oxygen setpoints according to whether the gas oxygen sensor 12 or the dissolved oxygen sensor 28,30 is providing the feedback to the oxygen control loop 160. Oxygen setpoints for each culture chamber 36 can be entered to the oxygen servo control loop 160 manually by the operator one at a time at the operator interface 28, or multiple setpoints can be entered automatically according to the timing and sequence of a particular profile during-profile control. Profiles are the specific series of timed setpoints that can be created by the user at the operator interface 28, or can be created by recording 24 the gas oxygen tensions 120 that are generated as the result of direct control of the dissolved oxygen tension 280 while a dissolved oxygen sensor 28,30 is driving the oxygen control loop 160. Once developed, profiles can be stored and used repeatedly. The operator interface 28 may be built into the controller 16 on the front panel 220 or located on an optional remote computer monitor or other console 22 as depicted in FIGS. 2-6.

The controller 16 may or may not include other control or monitoring functions in addition to dynamic oxygen control, such as the control of temperature, humidity, CO₂, or other gases. Sensors for these are depicted schematically in FIG. 1 by 14. For example, since CO₂ is commonly employed in cell culture to maintain pH, it is likely any controlled atmosphere culture chamber 36, 10, 38, 110, 236, 238 with dynamic oxygen control will also need CO₂ control. This is depicted schematically in FIGS. 3-6 by a tank of CO₂ with a solid arrow representing a necessity for CO₂ control in these embodiments. However, the retrofit embodiment as depicted in FIG. 2 has a hatched arrow for the CO₂ tank to represent the optional inclusion of the CO₂ control function in the oxygen profile controller 16. Whether CO₂ control is necessary or not depends on the incubator 10. If the commercially available incubator 10 to be retrofitted with the oxygen profile controller 16 is a CO₂ incubator, the CO₂ control function will not be required in the oxygen profile controller 16 because it is already built into the incubator. However, if the commercially available incubator 10 is just a thermal incubator with no CO₂ control, then CO₂ control will be necessary in the oxygen profile controller 16. Temperature control involves similar considerations. Since temperature control is commonly necessary, some embodiments will require temperature control built into the oxygen profile controller as well, such as those embodiments depicted in FIGS. 5 and 6. However, if the culture chamber is a commercially available incubator 10 with temperature control as depicted in FIG. 2, or if the culture chambers are sub-chambers 36, 38 inserted into a temperature controlled host chamber 360 as depicted in FIGS. 3 and 4, no temperature control will be required. Such additional control loops are depicted in FIG. 1 by bi-directional arrows 140, but will not be discussed further since they are extraneous to the primary invention of dynamic oxygen control.

In multiple chamber configurations as depicted in FIGS. 3, 4, and 6, the controller 16 controls oxygen and oxygen profiles and any other pertinent variables (e.g. CO₂, temperature, humidity) simultaneously but independently in each chamber. Similar or different oxygen tensions can be controlled in each chamber. The same or different profiles can be started and stopped at any time in any chamber without disturbing the other chambers. Only two chambers are illustrated here to represent multi-chamber systems, but multi-chamber systems can consist of any number of chambers. Also, any combination of chambers with gas only, liquid only, or both gas and liquid sensors in the oxygen control loop 160 can be configured into such multi-chamber systems.

With reference now specifically to FIGS. 2-6, these illustrate various different physical embodiments of the invention. The functional elements that have already been described and shown in FIG. 1 are identified with the same reference numbers in FIGS. 2-6, and a detailed discussion of them need not be repeated here. Also, regardless of which physical embodiment is described in FIGS. 2-6, it should be presumed that each of the depicted culture chambers with dynamic non-static oxygen control may be equipped with the entire complement of functional components depicted in FIG. 1 or any sub-set of functions thereof. That is, any culture chamber in any physical embodiment may be equipped with a gas oxygen sensor only, or a dissolved oxygen sensor only, or both gas and dissolved oxygen sensors that can be switched back and forth. This is depicted in FIGS. 2-6 by arrows pointing from the culture chamber to the controller to signify the feedback component of the oxygen profile control loop, but no further distinction will be made. Also, the man-machine interface depicted in FIG. 1 by functional block 28, as schematized in FIGS. 2-6, is either built into the controller 16 on the front panel 220 or located on an optional remote computer monitor or other console 22, and need not be discussed further.

With reference now specifically to FIG. 2, this figure illustrates one physical embodiment in which the oxygen profile controller 16 is designed to retrofit an existing commercially available incubator 10 and control gas oxygen profiles in the entire incubator. If the incubator 10 is a CO₂ incubator with it's own CO₂ control, the oxygen profile controller 16 will not need to control CO₂. If the incubator 10 has no CO₂ control, the controller 16 may also need to control CO₂. While a single incubator 10 is shown here, it is possible to design one controller 16 that will control oxygen profiles independently but simultaneously in any number of incubators.

With reference now specifically to FIG. 3, another physical embodiment consists of one or more sub-chambers 36, 38 placed inside one or more temperature controlled host chambers 360. Only two sub-chambers are depicted here 36, 38, but any number of sub-chambers is possible. The host chamber 360 may be a commercially available thermal incubator, a commercially available CO₂ incubator with temperature control, a temperature controlled walk-in room, a temperature controlled water bath, or any other suitable host chamber with temperature control. The associated oxygen profile controller 16 controls the oxygen concentration independently but simultaneously in each sub-chamber 36, 38. In this case, CO₂ control for each sub-chamber may also be required, even where the sub-chambers 36, 38 are inserted into a CO₂ controlled incubator, because the sub-chamber atmospheres are isolated from the atmosphere of the host chamber 360.

FIG. 4 specifically illustrates yet another physical embodiment with dynamic non-static oxygen control that works by a method best described as remote conditioning. In this embodiment, gas is controlled remotely from the culture chamber(s) 36, 38 in a separate mixing chamber 40 or gas stream blender. The controlled gas is then flushed into the various culture chambers 36, 38 so as to condition their gas phase without actually having real-time gas control in any culture chamber. Conditioning can be continuous or intermittent. Dissolved oxygen sensors, if utilized, would be located in the culture chamber, but the gas oxygen sensor would be required in the mixing chamber 40 or blender. The controlled gas mixtures can be moved by means of a pump 42 or by means of simple pressure differentials. Furthermore, the controlled gas can be circulated back and forth between the mixing chamber/blender and a culture chamber, or it can be infused in one direction through the culture chamber and exhausted out. There may be one gas mixer/blender 40 for each separate culture chamber 36, or it may be possible to multiplex from one mixer/blender 40 to multiple culture chambers 36, 38.

With reference now specifically to FIGS. 5 and 6, FIG. 5 illustrates an embodiment best described as a completely integrated stand-alone oxygen-profiling incubator 110, with temperature, CO₂, and O₂ controls all built in. The oxygen profile controller 116 is built into the incubator cabinet. FIG. 6 illustrates a similar integrated stand-alone oxygen-profiling incubator 210, but here having more than one chamber 236, 238 and having a built-in oxygen profile controller 216 for controlling the dynamic non-static oxygen tensions in each chamber 236, 238 simultaneously but independently. While this illustration depicts two chambers, such an embodiment could have any number of chambers.

In operation in all the foregoing embodiments, open culture vessels 32,33 containing cultures are placed in the culture chamber 36, and control is initiated in the oxygen control loop 160 and all the other pertinent control loops 140. Over the duration of the culture, the gas phase oxygen concentration 120 is then raised or lowered as required to regulate the dissolved oxygen concentration 280 in the culture media at the desired levels. However, those actual gas oxygen tensions at those specific times that are required to achieve a desired result may not be obvious or straightforward. That is because the change dynamics of dissolved oxygen 280 as a function of the gaseous oxygen 120 in contact with the liquid is limited by the relatively slow rate of diffusion of oxygen in and out of the liquid phase, and throughout the depths of the medium. Superimposed on this is a potentially rapidly fluctuating oxygen consumption rate by the cells in the culture, which may be changing in number and metabolic activity. However, each specific culture preparation is likely to behave in a reproducible way. That is, if prepared identically, all will have the same diffusion constant as determined by the identical surface area of their gas/liquid interface, and the identical media depth. Plus, the same cells plated at the same density under the same conditions will usually proliferate and metabolize at roughly the same rate and thus create an equivalent demand for oxygen at each time point over the duration of the culture.

The best specific gas oxygen profile for any given culture preparation can be determined by culturing in a chamber 36 fitted with both a gas oxygen sensor 12 and a dissolved oxygen sensor 28,30 that is not disruptive to the culture of interest. First the oxygen control loop 160 is changed over via a switch 17 to receive feedback from the dissolved oxygen sensor 28,30. Then the desired dissolved oxygen setpoint(s) are entered to the oxygen control loop 160, and the culture is initiated. As the dissolved oxygen control loop works to control the dissolved oxygen tensions 280 by raising or lowering the gas oxygen tensions 120, the gas oxygen sensor 12 records the achieved gas oxygen levels 120 and time points associated with these levels since the initiation of the culture. This recording is then used to create a series of timed gas oxygen setpoints that will result in a gas profile that can sufficiently recreate those same dissolved oxygen levels in all those specific culture preparation with identical diffusion constants and cells.

If the dissolved oxygen sensor 28, 30 is disruptive to the culture of interest 33, it may be possible to use an extra expendable witness culture 32 to serve as a surrogate. This extra culture 32 may be an identical sample of the same culture preparation, or a culture preparation designed to be similar to the culture of interest.

Once the proper gas oxygen profile for a culture is known, and if the culture chamber 36 is fitted with a gas oxygen sensor 12, the gas oxygen sensor 12 may be used to provide feedback to the oxygen control loop 160 to control the gas oxygen setpoints in the profile. If the chamber also is fitted with a dissolved oxygen sensor 28, 30 and it is not disruptive to the culture, it will be possible to observe and record the dissolved oxygen concentration 280 to verify that the desired oxygen levels are achieved. If that chamber 36 does not have a dissolved oxygen sensor 28, 30 due to economic constraints, or if using the dissolved oxygen sensor 28, 30 is disruptive in any way to the culture of interest and therefore cannot be used due to technical constraints, the gas oxygen profile may blindly control the dissolved oxygen concentration 280. Furthermore, a gas oxygen profile may be used to control a fixed dissolved oxygen concentration 280 over the course of the culture, or a different profile may be used to deliberately change the dissolved oxygen concentration 280 over the course of the culture, according to the needs of the user.

If a chamber 36 is fitted with a dissolved oxygen sensor 28, 30 that is not disruptive in any significant way to the culture of interest, the dissolved oxygen sensor 28, 30 may be used to provide feedback to the oxygen control loop 160 and directly control oxygen setpoints designated as dissolved oxygen setpoints. A single dissolved oxygen setpoint may be used to keep the dissolved oxygen concentration 280 constant over the course of the culture, which is likely to but may not necessarily result in a dynamic change in the gas oxygen concentration 120 in order to do so. Alternately, a series of dissolved oxygen setpoints as specified in a dissolved oxygen profile may be controlled in order to deliberately change the dissolved oxygen concentration 280 over the course of a culture, which is likely to but may not necessarily result in a dynamic change in the gas oxygen concentration 120. If that chamber 36 also has a gas oxygen sensor 12, it will be possible to observe and record the gas oxygen level 120 over the course of the culture. As described previously, if that gas oxygen profile ever needs to be replicated, it can be converted to a series of gas oxygen setpoints in a designated gas oxygen profile. If that chamber 36 is not equipped with a gas oxygen sensor 12 due to economic constraints, it may blindly manipulate the gas oxygen levels 120 in order to control the dissolved oxygen concentration 280.

In addition to the incubators and growth chambers discussed above, this dynamic gas control and profiling can be used favorably in glove chambers, refrigerators, plant growth chambers, and any other enclosures in which open culture vessels are placed in order to indirectly control dissolved oxygen (or other gas) by exposure to the controlled atmosphere.

Although the preferred embodiment has been employed in connection with cell and tissue culture in open culture vessels consisting of plastic or glass plates, flasks, micro-wells, beakers, etc., it is possible to employ the principles of this invention in other applications where the concentration of a dissolved gas can be optimized over time by the dynamic change of the gas concentration in the gas phase that is in contact with the liquid.

While the invention has been described with reference to specific preferred embodiments, the invention is certainly not limited to these precise embodiments. Rather, many modifications and variations will become apparent to persons of skill in the art without departure from the scope and spirit of this invention, as defined in the appended claims.

I claim:

1. Method of building a profile of an environmental gas in terms of dissolved gas concentration over time for a culture chamber in which a tissue culture is processed in vitro in a medium under controlled conditions of temperature and gas pressures; the gas profile being based on the amount of gas dissolved in the medium; comprising
placing the tissue culture, in said medium, into said growth chamber;
controlling gas concentration of the gas in the chamber;
measuring continually the concentration of the gas dissolved in said medium while the tissue culture is in said culture chamber;
storing measurements of the dissolved gas concentration and corresponding measurement times; and
recording said measurements as a function of time to create said dissolved gas profile.

2. Method of building a profile according to claim 1 further comprising reiterating the steps of measuring, storing and plotting while adjusting the gas pressure of said environmental gas in accordance with measurements of dissolved gas from previous steps of measuring, storing, and plotting to arrive at an desired profile of dissolved gas concentration over time.

3. Method of building a profile according to claim 1 wherein said environmental gas is O₂.

4. Method of building a profile according to claim 1 wherein said environmental gas is CO₂.

5. Method of building a profile according to claim 1 wherein said environmental gas is NO.

6. Method of building a profile according to claim 1 wherein said environmental gas is CO.

7. Method of building a profile of an environmental gas in terms of gas concentration over time for a culture chamber in which a tissue culture is processed in vitro in a medium under controlled conditions of temperature and gas partial pressures; the gas profile being based on the amount of gas dissolved in the medium; comprising
placing the tissue culture, in said medium, into said growth chamber;
measuring continually the concentration of at least one dissolved gas in said medium while the tissue culture is in said chamber;
continually controlling the concentration of the at least one dissolved gas in the chamber, by changing the concentration of the environmental gas in said chamber by introducing additional of said gas or a diluent gas to keep the level of dissolved gas in the medium at a desired level;
measuring the concentration of the gas in the atmosphere within said chamber;
storing measurements of the concentration of the gas in the atmosphere within the chamber together with corresponding measurement times; and
recording said measurements as a function of time to create said gas profile.

8. Process according to claim 7 wherein said measuring of the concentration of the dissolved gas in the medium is carried out using a non-invasive dissolved gas sensor.

9. Process for optimizing the growth of tissue in a culture chamber in which gas concentrations of one or more environmental gases are controlled, comprising:
placing a tissue culture in a medium in said chamber and maintaining the tissue culture within the chamber over an incubation time;
programming a gas controller with a gas profile in which the concentration of said gas varies over time to achieve a desired condition of growth of said tissue culture; and
dynamically controlling said environmental gas within said growth chamber according to said gas profile.

10. Process according to claim 9 wherein said environmental gas is O₂.

11. Process according to claim 9 wherein said environmental gas is CO₂.

12. Process according to claim 9 wherein said dynamically controlling includes adjusting the relative amount of said environmental gas in said chamber at intervals during said incubation time.

13. Process according to claim 12 wherein said adjusting includes gradually ramping the concentration of said environmental gas in said chamber.

14. Process for optimizing viability of tissue in a culture chamber without tissue growth in which gas concentrations of one or more environmental gases are controlled, comprising:
placing a tissue culture in a medium in said chamber and maintaining the tissue culture within the chamber over an incubation time;
programming a gas controller with a gas profile in which the concentration of said gas varies over time to achieve a desired condition of growth of said tissue culture; and
dynamically controlling said environmental gas within said growth chamber according to said gas profile.

15. Process for simulating in vitro natural conditions that occur to a living tissue in vivo, with the tissue being in a culture chamber in which gas concentrations of one or more environmental gases are controlled, comprising:
placing the tissue culture in a medium in said chamber and maintaining the tissue culture within the chamber over an incubation time;
programming a gas controller with a gas profile in which the concentration of said gas varies over time to achieve a desired condition of growth of said tissue culture; and
dynamically controlling said environmental gas within said growth chamber according to said gas profile.

16. Process for controlling the growth of tissue in a culture chamber in which gas concentrations of one or more environmental gases are controlled, comprising:
placing a tissue culture in a medium in said chamber and maintaining the tissue culture within the chamber over an incubation time;
measuring continually the concentration of at least one dissolved gas in said medium while the tissue culture is in the chamber; and
automatically changing the concentration of said environmental gas within said growth chamber by employing the dissolved gas concentration as a feedback to maintain the dissolved gas at a desired level in said medium.
